(19) United States
(12) Patent Application Publication
(10) Pub. No.: US 2020/0121843 A1
(43) Pub. Date: Apr. 23, 2020

(54) PERITONEAL DIALYSIS SYSTEM

(71) Applicant: FRESENIUS MEDICAL CARE DEUTSCHLAND GMBH, Bad Homburg (DE)

(72) Inventor: Peter WABEL, Darmstadt (DE)

(73) Assignee: FRESENIUS MEDICAL CARE DEUTSCHLAND GMBH, Bad Homburg (DE)

(21) Appl. No.: 16/626,921

(22) PCT Filed: Jun. 28, 2018
(86) PCT No.: PCT/EP2018/067436

§ 371 Date: Dec. 27, 2019

(30) Foreign Application Priority Data
Jun. 28, 2017 (DE) 10 2017 114 401.3

(51) Int. Cl. A61M 1/28

(52) U.S. Cl. CPC A61M 1/287; A61M 2205/6018; A61M 2205/6072

(57) ABSTRACT

The present invention relates to a peritoneal dialysis system comprising at least one peritoneal dialysis machine, wherein the peritoneal dialysis system furthermore has at least one water unit for preparing and/or metering water and at least one concentrate container, wherein the concentrate contains has at least one connector or is in communication therewith and wherein the water unit and/or the peritoneal dialysis machine has at least one receiver for the connector, wherein the connector has at least one piece of information, preferably in the form of a barcode, that represents the quantity of the water to be filled into the concentrate container or enables conclusions thereon, and wherein the water unit or the peritoneal dialysis machine is configured to read this information and to carry out or initiate the addition of water in accordance with the information read.

The present invention relates to a peritoneal dialysis system comprising at least one peritoneal dialysis machine.

Peritoneal dialysis is carried out by patients at a typical treatment site.

It can be a medical practice or clinic. Peritoneal dialysis frequently takes place at home or in the home bedroom. Solution bags of fresh dialysis solution are used as part of the peritoneal dialysis treatment; said dialysis solution is introduced via a hose system and a patient catheter into the abdominal cavity of the patient and remains there for a specific time duration. The peritoneum acts as a dialysis membrane that enables an exchange between the dialysis solution and the blood of the patient in the form that wastes are transferred from the blood of the patient via the membrane into the dialysis solution. The dialysis solution is drained from the abdominal cavity again after a specific dwell time.

There is in particular a greater logistical effort in those cases in which the patient carries out the treatment at home in that solution bags have to be transported home and have to be stored in sufficient numbers there. There is a further disadvantage in that the manufacture of the solution bags is associated with a certain expense, which makes their provision more expensive for the patient.

It is therefore the underlying object of the present invention to provide a peritoneal dialysis system by which the peritoneal dialysis treatment can also be carried out at home with a comparatively small logistical effort and inexpensively.

This object is achieved by a peritoneal dialysis system having the features of claim 1.

Provision is accordingly made that the peritoneal dialysis system furthermore has at least one water unit for preparing and/or metering water and at least one concentrate container, wherein the concentrate container has at least one connector or is in communication therewith and wherein the water unit and/or the peritoneal dialysis machine has at least one receiver for the connector wherein the connector has at least one piece of information, preferably in the form of a barcode, that represents the quantity of the water to be filled into the concentrate container or enables conclusions thereon, and wherein the water unit or the peritoneal dialysis machine is configured to read this information and to carry out or initiate the addition of water in accordance with the information read.

Provision is thus made in accordance with the invention that water is filled into the concentrate container or containers by means of the water unit and that the solution containers suitable for the treatment are obtained in this manner and the ready-to-use dialysis solution or a portion thereof is prepared. This means that such solution containers do not have to be transported to the patient, but that the provision of the concentrate containers at the point of care (POC) is rather sufficient, with the latter being able to be a room at the patient's home, for example. The completed dialysis solution is only prepared there. This facilitates the transport and the storage since the concentrate containers take up substantially less space and are lighter and less expensive than the completed solution bags or other solution containers.

The term “container” within the framework of the present invention includes all conceivable receptacles such as those with fixed walls such as boxes or also those with flexible walls such as bags.

The information on how much water has to be filled into the concentrate container is located on the connector or connectors that can be arranged directly on the concentrate container, but is preferably at the end of a hose that is connected to the inner space of the concentrate container. In this respect, the information can specifically include the quantity (mass, volume, etc.) of the water or can also only permit indirect conclusions thereon. This can take place, for example, such that the information only includes the type of the concentrate and the quantity of water that is to be added to the concentrate to obtain the completed dialysis solution or a portion thereof is read from such information e.g. from a storage medium.

Alternatively, other fluids such as electrolyte solutions can also be used for preparing the completed dialysis solution.

The “completed dialysis solution” is to be understood as the dialysis solution that can be supplied to the patient. A “portion of the completed dialysis solution” is to be understood such that the completed dialysis solution is mixed e.g. in the peritoneal dialysis machine from two or more portions, i.e. in this case, the completed dialysis solution is only prepared in the peritoneal dialysis machine from the portions of the completed dialysis solution.

The water unit is preferably configured to be connected to a conventional domestic water system or to a conventional faucet.

It is alternatively also conceivable to provide a different fluid preparation device, e.g. in the form of a water tank or a fluid tank, instead of a faucet.

The water unit can e.g. comprise at least one RO unit and/or one or more filters. Purified water is prepared from mains water by reverse osmosis in the RO unit. A further purification is possible by a further treatment of water in one or more filters. They can be connected upstream and/or downstream of the RO unit.

Instead of an RO unit, a different unit can also be present to prepare pure water such as a unit for deionization, for distillation or also a combination of one or more of the aforesaid units.

It is conceivable that the concentrate container is a concentrate bag and/or that at least one hose on whose end the connector is arranged is located at the concentrate container. The hose or hoses are in fluid communication with the inner space of the concentrate container or containers so that water can be filled into the concentrate container through the hose and the completed dialysis solution or a portion thereof can be drained via said hose or via a different hose from the concentrate container to the peritoneal dialysis machine.

Provision is preferably made that the concentrate container has at least two connectors or is in communication therewith, wherein at least one of the connectors includes the named piece of information and the other connector includes the same information or a different piece of information and wherein both the peritoneal dialysis machine and the water unit each have at least one receiver for a respective one of these connectors.

A preferred embodiment of the invention thus comprises the concentrate container being designed with at least two hoses of which each is provided with a connector. One connector serves the connection to the water unit and one connector serves the connection to the peritoneal dialysis machine.

At least one of these connectors includes the above-named piece of information that represents the quantity of the water to be filled into the concentrate container or enables conclusions thereon. This information can be arranged on the connector e.g. in the form of a barcode or also in the form of a different code that can be read. The water unit and/or the peritoneal dialysis machine include at least one receiver for receiving this connector and reading means that are configured to read the information on the connector.

If the water unit includes this receiver and if the connector is plugged therein, the water unit can directly provide the desired quantity of water and meter it into the concentrate container.

If the peritoneal dialysis machine includes this receiver and if the connector is plugged therein, the peritoneal dialysis machine transmits the information to the water unit so that it can provide the desired quantity of water and can meter it into the concentrate container.

It is also conceivable in an alternative embodiment that the metering of the quantity of water takes place by the peritoneal dialysis machine.

The other connector can likewise be a connector provided with a piece of information or can also be a standard connector that does not include any information. The other connector can, however, likewise be a connector including a piece of information, e.g. in the form of a barcode or in the form of a different code. Both connectors can include the same information or different information.

The concentrate container or containers preferably have all the substances that are required for peritoneal dialysis and that are present in the desired concentration after the dilution with water, with the substances comprising the following group: electrolyte ions and at least one osmotic agent, in particular glucose, and optionally one or more buffers. The concentrate or concentrates can in this respect be concentrated solutions and dry concentrates.

The peritoneal dialysis machine and/or the water unit can have a plurality of receivers for connectors of different concentrate containers and/or the peritoneal dialysis machine can have at least one receiver for a connector for a prefilled solution container.

If a plurality of receivers are present, a plurality of concentrate containers can be connected and can be consecutively or also simultaneously filled with water. The concentrate containers can include identical concentrates or different concentrates.

It is also conceivable that at least one receiver for a standard connector of a completed solution container is present so that such solution containers already containing dialysis solutions can also be connected.

The concentrate container or containers can have a first hose by means of which water can be added from the water unit into the concentrate container and can have a second hose by means of which the concentrate prepared in the concentrate container can be supplied to the peritoneal dialysis machine. The first hose can be provided with a code, in particular a barcode, including the piece of information on the quantity of water to be added and can be introduced into the water unit; the other hose can be configured with a standard connector that is connected to the peritoneal dialysis machine. This hose can, however, also be provided with a connector having a piece of information, e.g. on the type and quantity of the solution.

It is, however, also conceivable that the concentrate container or containers only have one single hose by means of which water can be added into the concentrate container from the water unit and by means of which the concentrate prepared in the concentrate container can be supplied to the peritoneal dialysis machine.

It is furthermore conceivable that the concentrate container or containers has/have at least one hose by means of which water can be added into the concentrate container from the water unit, with at least one filter and/or at least one one-way valve being in said hose. The filter effects a further purification of the water by means of which the concentrate is diluted; the one-way valve ensures that no fluid flows from the concentrate container back to the water unit, but only in the opposite direction.

The filter and/or the one-way valve can be configured as disposables.

The peritoneal dialysis machine can comprise at least one receiver for the connector having the information and the peritoneal dialysis machine can furthermore have transmission means to transmit this information to the water unit. This is necessary when the connector is plugged into the peritoneal dialysis machine, but not into the water unit. In this case, receivers for connectors and a reading unit of the pieces of connector information can be dispensed with in the water unit.

These transmission means can be configured to transmit the information in a wired or wireless manner.

The connector or connectors can have one or more pieces of information on the type of the concentrate, in particular on the ingredients and/or their concentrations.

The concentrate or concentrates can be present in liquid or solid form in the concentrate container or containers.

The water unit can have at least one balancing pump. This introduces water into the concentrate container or containers in the desired quantity.

Instead of a pump, any desired other metering unit can also be provided that meters the water, preferably with a precision of 1%.

The water unit and/or the peritoneal dialysis machine can be arranged on a movable rack or trolley or also in a receptacle that simultaneously serves as a base for the peritoneal dialysis machine.

The peritoneal dialysis machine is preferably an APD machine, but any other desired peritoneal dialysis machines can also be used within the framework of the invention.

If a plurality of concentrate containers are connected, they can have different volumes or the same volumes.

Provision can generally be made that the volume of the concentrate container represents a piece of information that is likewise included in the code, in particular the barcode, of the connector. Exemplary volumes are 2 l, 5 l or 15 l, if only one bag is to be used for the total therapy.

The water unit can, for example, be connected to a platform and/or to the peritoneal dialysis machine wirelessly, via WiFi, Bluetooth or via other techniques.

It is also possible that at least one scale is provided for detecting the weight of the concentrate container and that communication means for communication between the scale and the water unit are present via which the information on the current weight of the concentrate container can be transmitted to the water unit. It is thus possible to use a non-balancing pump. In this case, the filling volume in the concentrate container is defined via a scale. It is conceivable that the scale or another unit connected thereto controls a valve that blocks or releases the flow path in the concentrate container depending on whether the desired volume or weight has already been reached in the concentrate container. It is thus also possible to take care of or verify a plurality of concentrate containers after one another.

Provision is made in an embodiment that the peritoneal dialysis machine has at least one memory in which the prescription is stored which is to be carried out for the treatment. Based on the prescription, the peritoneal dialysis machine determines which concentrate containers are to be connected to the water unit. The water unit reads the information from the barcodes or other codes as to what volume amount is to be used to dilute the concentrates and carries out the corresponding filling of the concentrate container or containers.

The water unit can have means that are configured to issue a time stamp from which the time when the solution would be completely diluted results. This time stamp is communicated to the peritoneal dialysis machine via a connection (e.g. wireless WLAN or LAN). The peritoneal dialysis machine verifies whether the time stamp is still valid, i.e. that too long a time duration has not elapsed since the preparation of the solution and only admits the solution for the treatment when the time duration has not yet been exceeded. Typical values are in the range from 6 to 24 h.

For a better intermixing of the solutions, at least one movement element can be provided, such as a shaker, to move the concentrate container or containers.

It is alternatively or additionally possible to apply pressure surges on the filling of the concentrate container to ensure a better intermixing.

The alternating use of suction phases and filling phases is also possible.

The present invention furthermore relates to a concentrate container including a concentrate that is used after dilution for use as a solution for peritoneal dialysis, wherein the concentrate container has at least one connector that has one or more pieces of information, preferably in the form of at least one barcode, that represents the quantity of the water to be filled into the concentrate container or enables conclusions thereon. The concentrate container can have one or two hoses, for example. In the case of two hoses, each hose can be provided with a connector that has a piece of information or only one of the connectors, as was stated in more detail above.

The present invention furthermore relates to a method of preparing a peritoneal dialysis solution, wherein the preparation takes place using a peritoneal dialysis system in accordance with one of the claims 1 to 12.

The method is preferably carried out such that it comprises the step of introducing the at least one connector into the water unit and/or into the peritoneal dialysis machine, that the information located on the connector is read and that the addition of water into the concentrate container or containers takes place in accordance with the information read.

Further details and advantages of the invention will be explained in more detail with reference to an embodiment shown in the drawing.

There are shown:

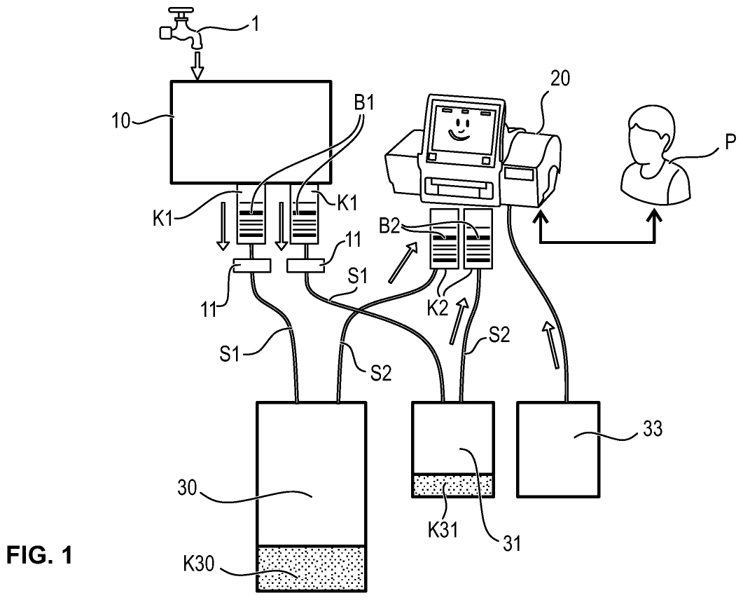

FIG. 1: a schematic view of the peritoneal dialysis system having two hoses with connector each per bag;

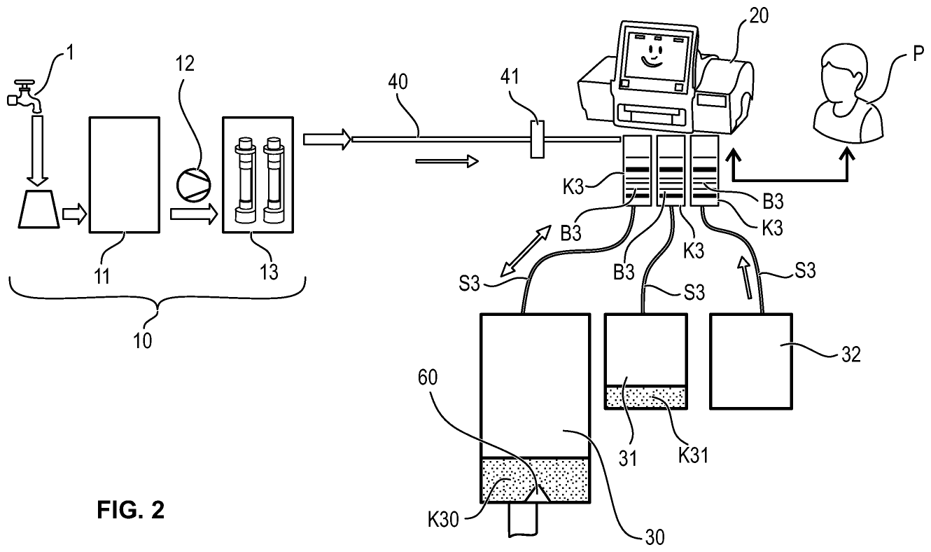

FIG. 2: a schematic view of the peritoneal dialysis system having one respective hose with connector per bag; and

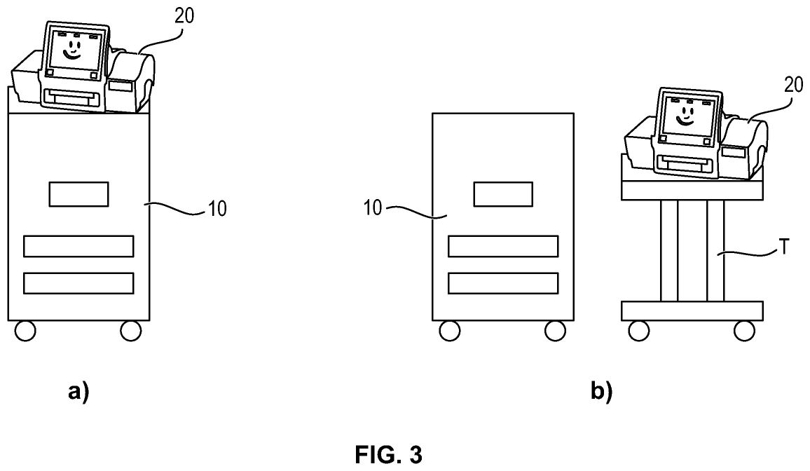

FIG. 3: a schematic view of the peritoneal dialysis system with trolley.

FIG. 1 shows a peritoneal dialysis system in accordance with the invention in a first embodiment.

The water unit that comprises means for metering and means for treating or purifying the water is marked by reference numeral 10.

The water unit 10 is connected to a faucet 1 of a home or of a clinic, etc.

Reference numeral 20 denotes a peritoneal dialysis machine; the reference symbol P denotes a patient whose prescription is stored in the peritoneal dialysis machine 20.

Two concentrate containers of different sizes and having the concentrates K30 and K31 are marked by reference numerals 30 and 31, with the concentrate container 30 having a volume of 15 l and the concentrate container 31 having a volume of 5 l.

Both are prefilled with a concentrate K30, K31 that is diluted with water from the unit 10.

As can be seen from FIG. 1, each of these concentrate containers 30, 31 has a first hose S1 having a first connector K1 and a second hose S2 having a second connector K2, with the hoses S1 serving the supply of water from the unit 10 into the bags 30, 31 and the hoses S2 the drainage of the diluted concentrate or the completed dialysis solution from the bags 30, 31 to the peritoneal dialysis machine 20. A respective one-way ultrapure filter 11 is located in the hoses S1.

The connectors K1 and K2 each include barcodes B1 and B2 respectively.

The barcodes B1 include information that permits conclusions on the volume or mass of water that is to be filled into the bags 30, 31. The water unit 10 reads this information and meters a corresponding amount of water into the bags so that the desired concentration of the individual substances is present in the bags.

As soon as this is the case, the solution can be used for use in the peritoneal dialysis machine 20. The peritoneal dialysis machine 20 reads which solution it is from the barcode B2 of the connector K2.

It is possible by the present invention to dispense with the delivery of solution bags that contain the completed dialysis solution. The provision of concentrate bags or concentrate containers whose contents are only diluted to the desired concentrations on site is rather sufficient.

The peritoneal dialysis machine 20 likewise has one or more receivers for standard connectors so that a completely filled standard solution bag 33 can also be connected and used, as is shown in FIG. 1.

FIG. 2 shows an embodiment in which the concentrate containers only have one hose S3 that is designed with a connector K3 having a barcode B3. In another respect, parts that are the same or have the same function are marked by the same reference numerals as in FIG. 1.

The connector K3 is introduced into a receiver of the peritoneal dialysis machine 20 and is thus not in direct communication with the water unit 10.

The water unit 10 comprises a prefilter and an RO unit 11, a pump such as an impeller pump 12 and filters 13 connected downstream thereof. The water unit 10 is in communication with the peritoneal dialysis machine 20 via a hose 40 or via another line. A further filter 41, e.g. an 0.2μ filter, is located in this hose 40.

The peritoneal dialysis machine 20 reads the barcodes B3 and reports what amount of water is to be metered to the water unit 10. The water unit 10 delivers this amount of water to the peritoneal dialysis machine 20 and from it into the concentrate containers 30, 31. After the dilution and dissolving into the concentrate containers 30, 31 has taken place, the solution is delivered to the peritoneal dialysis machine or is conveyed from it to the patient, e.g. by means of one or more pumps. Reference numeral 60 marks a safety chamber in the concentrate container 30.

The barcodes B1 and B3 comprise information that indicates the type of solution to be prepared or the type of concentrate and the quantity of the water to be added. Despite the same reference symbol B1, the barcode B1 of the bag 30 optionally has a different piece of information than the barcode B1 of the bag 31. This applies accordingly to the barcodes B2 and B3.

The peritoneal dialysis system can be used as a batch-type system that prepares the solutions for the respective treatment batch-wise.

It is conceivable to move the peritoneal dialysis machine or the peritoneal dialysis system as a whole to the patient.

The peritoneal dialysis machine can be moved on a trolley T from a filling station or from the water unit to the patient.

FIG. 3a) shows the peritoneal dialysis machine 20 and the water unit as a movable peritoneal dialysis system, wherein the peritoneal dialysis machine 20 stands on top of the water unit 10.

FIG. 3b) shows the peritoneal dialysis machine 20 on a trolley T and independently of the water unit 10 that is configured in this case as a separate unit separated from the peritoneal dialysis machine 20.

1. A peritoneal dialysis system comprising at least one peritoneal dialysis machine, characterized in that the peritoneal dialysis system has at least one water unit for preparing and/or metering water and at least one concentrate container, wherein the concentrate container has at least one connector or is in communication therewith and wherein the water unit and/or the peritoneal dialysis machine has at least one receiver for the connector, wherein the connector has at least one piece of information, preferably in the form of a barcode, that represents the quantity of the water to be filled into the concentrate container or enables conclusions thereon, and wherein the water unit or the peritoneal dialysis machine is configured to read this information and to carry out or initiate the addition of water in accordance with the information read.

2. A peritoneal dialysis system in accordance with claim 1, characterized in that the concentrate container is a concentrate bag; and/or in that at least one hose on whose end the connector is arranged is located at the concentrate container.

3. A peritoneal dialysis system in accordance with claim 1, characterized in that the concentrate container has at least two connectors or is in communication therewith, wherein at least one of the connectors includes the named piece of information and the other connector includes the same information or a different piece of information and wherein both the peritoneal dialysis machine and the water unit each have at least one receiver for a respective one of these connectors.

4. A peritoneal dialysis system in accordance with claim 1, characterized in that the concentrate container or containers has/have all the substances that are required for peritoneal dialysis and that are present in the desired concentration after the dilution with water, with the substances comprising the following group: Electrolyte ions and at least one osmotic agent, in particular glucose.

5. A peritoneal dialysis system in accordance with claim 1, characterized in that the peritoneal dialysis machine and/or the water unit has/have a plurality of receivers for connectors of different concentrate containers; and/or in that the peritoneal dialysis machine has at least one receiver for a connector for a prefilled solution container.

6. A peritoneal dialysis system in accordance with claim 1, characterized in that the concentrate container or containers has/have a first hose by means of which water can be added into the concentrate container from the water unit and a second hose by means of which the concentrate prepared in the concentrate container can be supplied to the peritoneal dialysis machine; or in that the concentration container or containers has/have only one hose by means of which water can be added into the concentrate container from the water unit and by means of which the concentrate prepared in the concentrate container can be supplied to the peritoneal dialysis machine.

7. A peritoneal dialysis system in accordance with claim 1, characterized in that the concentrate container or containers has/have at least one hose by means of which water can be added into the concentrate container from the water unit, with at least one filter and/or at least one one-way valve being located in said hose.

8. A peritoneal dialysis system in accordance with claim 1, characterized in that the peritoneal dialysis machine comprises at least one receiver for the connector having the information and the peritoneal dialysis machine furthermore has transmission means to transmit this information to the water unit.

9. A peritoneal dialysis system in accordance with claim 8, characterized in that the transmission means are configured to transmit the information in a wired or wireless manner.

10. A peritoneal dialysis system in accordance with claim 1, characterized in that the connector or connectors has one or more pieces of information on the type of the concentrate, in particular on the ingredients and/or their concentrations.

11. A peritoneal dialysis system in accordance with claim 1, characterized in that the water unit has at least one balancing pump; and/or in that at least one scale is provided for detecting the weight of the concentrate container; and in that communication means are present for communication between the scale and the water unit via which the information on the current weight of the concentrate container can be transmitted to the water unit.

12. A peritoneal dialysis system in accordance with claim 1, characterized in that at least one movement element is provided to move the concentrate container for a better dissolving of the concentrate; and/or in that means are present to produce pressure surges in the concentrate container.

13. A concentrate container including a concentrate that is used after dilution for use as a solution for peritoneal dialysis, wherein the concentrate container has at least one connector that has one or more pieces of information, preferably in the form of at least one barcode, that represents the quantity of the water to be filled into the concentrate container or enables conclusions thereon.

14. A method of establishing a peritoneal dialysis solution, characterized in that the establishing takes place using a peritoneal dialysis system in accordance with claim 1.

15. A method in accordance with claim 14, characterized in that the method comprises the step of introducing the at least one connector into the water unit and/or into the peritoneal dialysis machine, that the information located on the connector is read and that the addition of water into the concentrate container takes place in accordance with the information read.
